SEQUENCE LISTING (11 sequences)

SEQ ID NO: 1 - RNA, 21 nt, Artificial sequence
  Single strand RNA oligonucleotide, used as a part of double stran ded interfering RNA
augaucuacccgccucauguu

SEQ ID NO: 2 - RNA, 21 nt, Artificial sequence
  Single strand RNA oligonucleotide, used as a part of double stran ded interfering RNA
caugaggcggguagaucauuu

SEQ ID NO: 3 - RNA, 21 nt, Artificial sequence
  Single strand RNA oligonucleotide, used as a part of double stran ded interfering RNA
caguugcgcagccugaauguu

SEQ ID NO: 4 - RNA, 21 nt, Artificial sequence
  Single strand RNA oligonucleotide, used as a part of double stran ded interfering RNA
cauucaggccaggcaacuguu

SEQ ID NO: 5 - RNA, 21 nt, Artificial sequence
  Single strand RNA oligonucleotide, used as a part of double stran ded interfering RNA
ucuuauuauaccagagcucuu

SEQ ID NO: 6 - RNA, 21 nt, Artificial sequence
  Single strand RNA oligonucleotide, used as a part of double stran ded interfering RNA
gagcucugguauaauaagagg

SEQ ID NO: 7 - RNA, 21 nt, Artificial sequence
  Single strand RNA oligonucleotide, used as a part of double stran ded interfering RNA
ccagcacuucacccaucaauu

SEQ ID NO: 8 - RNA, 20 nt, Artificial sequence
  Single strand RNA oligonucleotide, used as a part of double stran ded interfering RNA
ugaugggugaagugcugggc

SEQ ID NO: 9 - RNA, 20 nt, Artificial sequence
  Single strand RNA oligonucleotide, used as a part of double stran ded interfering RNA
agacagcauaagcugcgccu

SEQ ID NO: 10 - RNA, 21 nt, Artificial sequence
  Single strand RNA oligonucleotide, used as a part of double stran ded interfering RNA
ggcgcagcuuaugcugucucu

SEQ ID NO: 11 - DNA, 3489 bp, Bos taurus
tgaacgtcagaggacgccccggaactccgcaccgccctcctccggccccgcagctccgatccgcgcacctccacgcctccgccggccccgcgccccgcccgcagccgagatgctcgcccgggccctgctgctctgcgctgccgtggcgctcagcggtgcagcaaatccttgctgttcccatccatgtcagaatcgaggtgtatgtatgagtgtaggatttgaccagtataaatgtgactgtacccgaacaggattctacggtgaaaactgtaccacacccgaatttctgacaagaataaaattactcctgaaacccactcccaacacagtgcactacatacttacccacttcaaaggagtctggaacattgtcaataagatctccttcctgcgaaatatgattatgagatatgtgttgacgtcgagatcacatttgattgagagtccaccaacttataatgtgcactacagctataaaagctgggaagccttttctaacctgtcttattataccagagctcttcctcctgtgcctgatgactgcccaacacccatgggtgtgaaagggaggaaagagcttcctgattcaaaagaagttgtgaaaaaagtacttctaagaagaaagttcattcctgatccccagggcacaaatctgatgtttgcattctttgcccagcacttcacccatcaatttttcaagacagattttgaacgaggaccagctttcactaagggaaagaaccatggggtggacttaagtcacatttatggtgaatctttagagagacagcataagctgcgccttttcaaggatggaaaaatgaaatatcagatgattaatggagagatgtatcctcccacagtcaaagatactcaggtcgaaatgatctacccgcctcatgttcctgaacacttgaagtttgctgtgggccaggaagtctttggtctggtgcctggtctgatgatgtatgccaccatttggctacgggaacacaacagagtgtgtgatgtgcttaaacaagagcatccagaatggggcgatgagcagttgttccagacaagcaggctaatcctgataggagaaactattaagattgtgattgaagactacgtacagcacttgagtggctatcacttcaaactgaagtttgacccagagctgcttttcaaccaacagttccagtaccagaaccgtattgctgctgagtttaacacgctctaccactggcatccccttctgcctgacgtctttcagattgatggtcaggagtacaactatcagcagtttatctataacaactctgtcttactggaacatggtctcactcagtttgttgaatcattcaccaggcaaagggctggcagggtcgctggcggtaggaatcttccagtcgcagtagagaaagtatcaaaggcttcaattgaccagagcagagagatgaaataccagtcttttaatgagtatcgcaaacgttttctcgtgaagccctatgaatcatttgaggaacttacaggagagaaggaaatggctgcagagttagaagcgctctatggagacatagatgccatggagttttatcccgcccttctggtagagaagccccgtccagacgccatctttggggagaccatggtagaagctggagcaccattctccctgaaaggacttatgggtaatcctatatgctctcccgagtactggaagcctagcactttcggtggagaagtaggttttaaaatcatcaacactgcctcaattcagtctctcatctgcagtaacgtgaaaggctgtccctttacctcattcagtgttcaagatacgcacctcaccaaaacggtcaccattaatgcaagctcttcccactctggactagatgatatcaaccccacagtcctactaaaggaacgttcaaccgaactgtagaagcctagcagtcatatttatttatttatatgaactgtttcttttaacttaattatttaatatttatatgaaactccttgtgttacttaacatcttctgttaaggagaaaaagggggtcatgcttgtgaagattttcatgttgattttaaagatgtcgaagtttcttcaagttaaaggggaaagcagttttcattcttctgtacaatccaatgggaaatgagtatgacattttttacttgaatttcaacttataataagaacaaaagctaagtttgaacatgtaagtgctgttgcaagatgacaaaatgctgcacatttcgacactatcatatttccagggtgtctcctatgatgcctgagaaacagctgtctactcactggtccttttcagtctccttttagccattttcagatcagtttacttcgactattttgttttcctggttttaaggtctgagtgtgtttttttggactctgcctgtactttcttacctgaacttatgcaagttttcaggaaatcctcagctcaaaactactacaaaaggcctttacaaaaaggtatacattcattttaagtgaaaggcaaagaactttacaaataaactataacctgattaagagcccaataccttaaagctctagggggttctcgacaccaagaacgtattcctattctaattaatgtctttcctcatttaaaagcaaataattgctgaatagttcccagggagacaatgcttcttttccacatctcattgtcagttgacattttctggtactgtatattaatttattgagagctattatgtcttcttaggatgactattataaactgggtttaaggctacatgatgttctttgttggcattatgtcagaatcagtgtatcttgtgggattacctctccaaattattactataacattagtgtttggattaagatttgtgaaaacatgtgaggaatccaactctggtatactgagatggaaagttggaattcgcctttaaggcttacctactcaccagcccacagagaacgttgtctcgttaccctggatgtgctgacactgacagtgtgtacgtttttgaaggacttgtggatgtttcattaattcattcacccctaacttctggagaaacattctttacaaagcactgtgggttcttaatatttttaaatcatgcactgaaaatcagtatttatgtaaataattgaacaggtatgcctgttaggcaaagggagaaaaatgaaatttcattaaagagaaataactcaggggaatttttaggattttatgtttaaatgatttatggttaataaagagtcaatagtagaagagcttgtattaaaaaaactgttaccttcattgatttttttaaaaaactgatttgttaaatatctgaatgactgaatctgggaatttggaatatatgagtgttttggtgcctcagactatggttaaattaatttatgtaaactgtaagtgttgaaacaaatagctgtttatttttgtattatttaaaaattgaaaacctcttctaaaataaattttgactgtttctgt

(19) United States
(12) Patent Application Publication
(10) Pub. No.: US 2004/0110698 A1
(43) Pub. Date: Jun. 10, 2004

(54) Oligonucleotides and methods using same for treating cox-ll associated diseases

(75) Inventors: Mordechai Shemesh, Raanana (IL); Laurence Shore, Rehovot (IL); Yehuda Stram, Nes Ziona (IL); Shulamit Michaeli, Kiryat Ono (IL); Haim Breitbart, Petah Tikva (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignees: Kimron Veterinary Institute; Bar-llan University

(21) Appl. No.: 10/315,218

(22) Filed: Dec. 10, 2002

(51) Int. Cl. A61K 48/00; C07H 21/04

(52) U.S. Cl. 514/44; 536/23.2

(57) ABSTRACT

A small interfering duplex oligonucleotide comprising a 15 to 30 base pair sequence being at least 90 % identical to a contiguous nucleic acid sequence of COX-II, as determined using the GCG BestFit software of the Wisconsin sequence analysis package, utilizing the Smith and Waterman algorithm, where gap weight equals 50, length weight equals 3, average match equals 10 and average mismatch equals −9.

### FIELD AND BACKGROUND OF THE INVENTION

The present invention relates to oligonucleotides which can be used to treat COX-II associated diseases such as, diabetes, stroke and Alzheimer's disease.

Arachidonic acid metabolites, collectively referred to as eicosanoids, such as hydroxyeicosatertraenoic acids, prostaglandins (PGs), thromboxane (TXs) and leucotrienes (LTs) play a central role in many diverse physiological and pathophysiological functions, including inflammatory responses, premature labor, cell proliferative disorders and stroke [Williams and DeBois (1996) Am. J. Physiol. 270:G393-400; Feng et al. (1993) Arch. Biochem. Biophys. 307:361-8].

Eicosanoids are produced by the catalytic activity of cyclooxygenase (COX) enzymes on arachidonic acid substrate. While in nascent cells, nearly all arachidonic acid is esterified in membrane phospholipids and is unavailable for eicosanoid biosynthesis, upon an extracellular stimulus, phospholipase A₂ cleaves arachidonic acid from the phospholipid, thereby availing cyclooxygenase enzymes of arachidonic acid for conversion to prostaglandins.

Two forms of COX have been described; a constitutive enzyme (COX-I) present in most cells and tissues and an inducible isoenzyme (COX-II) mainly expressed in response to cytokines, growth factors and other stimuli [Wilson (1991) J. Clin. Gastroenterol 13:S65-71; Appleton (1995) J. Pathol. 176:413-20]. These COX-Isoforms have both overlapping, as well as distinct physiological and pathophysiological functions. While COX-I is involved in the homeostasis of various physiological functions, COX-II is responsible for many pathological processes such as inflammation and cancer [Kirikitara et al. (1998) J. Exp. Med. 187:517-23; Reuter (1996) J. Clin. Invest. 98: 2076-85 and Brzozowski (1999) Eur. J. Pharmacol. 385:47-61]. For example, COX-II mRNA and protein levels are markedly increased in human colorectal adenocarcinomas relative to normal colonic mucosa and overexpression of COX-II has been identified as an early central event in colon carcinogenesis [Turini and DuBois (2002) 53:35-57]. By contrast, COX-I is the predominant isoform expressed in the normal gastrointestinal tract and kidney and has therefore been suggested to produce PGs important for maintenance of mucosal integrity and renal blood flow [Wilson (1991) J. Clin. Gastroenterol 13:S65-71].

Given the broad role of PGs in human physiology, it is not surprising that systemic suppression of PGs leads to undesired side effects. Thus, common non-steroidal antiinflammatory drugs (NSAIDs) that are active in reducing the prostaglandin-induced pain and swelling associated with the inflammation process are also active in affecting other prostaglandin-regulated processes not associated with the inflammation process. Use of high doses of most common NSAIDs can produce severe side effects, including life threatening ulcers, which limit their therapeutic potential [Murray and Brater (1993) Annu. Rev. Pharmacol. Toxicol. 33:345-65; Davies (1995) Dis. Colon Rectum 38:1311-21]. An alternative to NSAIDs is the use of corticosteroids, which have even more drastic side effects, especially when long term therapy is involved [Hanauer (2001) Rev. Gastroenterol. 1:169-76].

It is recognized that agents, which possess selective or specific inhibition of COX-II can be expected to provide improved gastrointestinal (GI) and renal safety while maintaining a high degree of anti-inflammatory, antipyretic and analgesic activity.

However, current available COX-II specific inhibitors exhibit poor selectivity in-vivo, inferior efficacy as compared to combined inhibition of COX-I and COX-II and enhanced ulceration and delayed healing when administered to subjects with pre-existing mucosal inflammation [Wallace et al. (1998) J. Clin. Gastroenterol. 27:S28-S34].

For example, Nabumetone and Nimesulide are presently marketed as selective COX-II inhibitors. Although they exhibits good selectivity in vitro (i.e., approximately 7 fold and 50 fold, respectively), they were shown to be non-selective in-vivo, when evaluated using whole-blood samples [Meade (1993) J. Biol. Chem. 268:6610-4; Partignani (1994) J. Pharmacol. Exp. Ther. 271:1705-12].

Several examples suggest that efficacy is not achieved using a number of commercially available COX-II inhibitors. Nimesulide, NS-398 and DuP697 were examined for their ability to reduce inflammation in rats. The drugs produced significant anti-inflammatory effect only when administered at doses, which were non-selective towards COX-II, as determined by whole blood thromboxane synthesis [Wallace (1998) Gastroenterology 115:1705-12].

Furthermore, L745,337 and NS-398, both of which are selective COX-II inhibitors, were shown to inhibit ulcer healing in rat models [Mizuno (1997) Gastroenterology 112:387-97; Schmassmann (1997) 112:A283]. These findings were further substantiated by the observation of Reuter and co-workers who showed in colitis rat model a suppressed colon PG synthesis upon administration of anti-inflammatory doses of a number of COX-II inhibitors (i.e., Diclofenac, naproxen, aspirin and L745,337), leading to profound exacerbation of the colitis and death [Reuter (1996) J. Clin. Invest. 98:2076-85]. Because such an exacerbation is likely to be due to a cross reaction of the inhibitors with COX-I, highly selective COX-II inhibitors are required.

An antisense inhibition may offer an added target selectivity. However most attempts to suppress COX-II activity via specific antisense oligonucleotides have failed [Yamada et al. (2000) Biochem. Biophys. Res. Comm. 269:415-421; Khan et al. (2001) Antisense and Nucleic Acid Drug Dev. 11:199-207, U.S. Pat. No. 6,344,323]. This may be explained mainly by technical obstacles pertaining to oligonucleotide uptake, availability of target site, and stability of the delivered nucleic acids. M-fold analysis data [Henry (1997) Anti Cancer Drug Design 12:1-14] demonstrated extensive secondary structures in the COX-II mRNA, which could explain the ineffectiveness of a large number of antisense oligonucleotides in-vivo. Furthermore, some of the COX-II specific antisense oligonucleotides mediated an undesirable increase in COX-II expression. The mechanism underlying this effect is yet to be elucidated; however it can involve the reversibility of the antisense effect.

There is thus a widely recognized need for, and it would be highly advantageous to have, oligonucleotides compounds and methods using same for treating COX-II associated diseases, which are devoid of the above limitations.

### SUMMARY OF THE INVENTION

According to one aspect of the present invention there is provided a small interfering duplex oligonucleotide comprising a 15 to 30 base pair sequence being at least 90% identical to a contiguous nucleic acid sequence of COX-II, as determined using the GCG BestFit software of the Wisconsin sequence analysis package, utilizing the Smith and Waterman algorithm, where gap weight equals 50, length weight equals 3, average match equals 10 and average mismatch equals −9.

According to another aspect of the present invention there is provided a pharmaceutical composition comprising the small interfering duplex oligonucleotide and a pharmaceutically acceptable carrier and/or diluent

According to yet another aspect of the present invention there is provided a method of reducing COX-II expression in a subject in need thereof, the method comprising administering to the subject a therapeutically effective amount of COX-II specific small interfering duplex oligonucleotide, thereby reducing COX-II expression.

According to still another aspect of the present invention there is provided a method of reducing COX-II mediated prostaglandin production in a subject in need thereof, the method comprising administering to the subject a therapeutically effective amount of COX-II specific small interfering duplex oligonucleotide for reducing COX-II expression, thereby reducing COX-II mediated prostaglandin production.

According to an additional aspect of the present invention there is provided a method of reducing COX-II expression in a subject in need thereof, the method comprising administering to the subject a therapeutically effective amount of at least one expressible polynucleotide encoding a COX-II specific small interfering duplex oligonucleotide, thereby reducing COX-II expression.

According to yet an additional aspect of the present invention there is provided a pharmaceutical composition for reducing COX-II-mediated prostaglandin production, the pharmaceutical composition comprising, as an active ingredient, a COX-II specific small interfering duplex oligonucleotide for reducing COX-II expression and COX-II-mediated prostaglandin production, and a pharmaceutically acceptable carrier.

According to still an additional aspect of the present invention there is provided use of COX-II specific small interfering duplex oligonucleotides for the manufacture of a medicament for the treatment and/or prevention of a medical condition whereby reducing COX-II expression is beneficial.

According to a further aspect of the present invention there is provided a pharmaceutical composition for reducing COX-II expression, the pharmaceutical composition comprising, as an active ingredient, a COX-II specific small interfering duplex oligonucleotide selected capable of reducing COX-II expression and a pharmaceutically acceptable carrier.

According to still further features in the described preferred embodiments the COX-II specific small interfering duplex oligonucleotide is selected incapable of reducing COX-I expression.

According to still further features in the described preferred embodiments the prostaglandin is selected from the group consisting of (PG)Gs, PGHs, PGIs, PGAs, PGBs, PGDs, PGEs and PGFs.

According to still further features in the described preferred embodiments the active ingredient is effective at a concentration of the small interfering duplex oligonucleotide between 5-15 μg/Kg body weight.

According to still further features in the described preferred embodiments the pharmaceutically acceptable carrier comprises lipomolecules.

According to still further features in the described preferred embodiments the lipomolecules are arranged in liposomes or micelles.

According to still further features in the described preferred embodiments the COX-II specific small interfering duplex oligonucleotide comprises at least one terminal 3′ hydroxyl group.

According to still further features in the described preferred embodiments the COX-II specific small interfering duplex oligonucleotide comprises blunt and/or overhanging ends.

According to still further features in the described preferred embodiments the overhanging ends comprise ends that are 1 to 6 nucleotides in length.

According to still further features in the described preferred embodiments the COX-II specific small interfering duplex oligonucleotide comprises ribonucleotides and/or ribonucleotide analogs.

According to still further features in the described preferred embodiments the COX-II specific small interfering duplex oligonucleotide is of between 15 to 30 base pairs.

According to still further features in the described preferred embodiments the COX-II specific small interfering duplex oligonucleotide is of between 18 to 25 base pairs.

According to still further features in the described preferred embodiments the COX-II specific small interfering duplex oligonucleotide is of between 21 to 23 base pairs.

According to still further features in the described preferred embodiments the COX-II specific small interfering duplex oligonucleotide is as set forth in SEQ ID NOs: 1-2, 5-10.

According to still further features in the described preferred embodiments the COX-II specific small interfering duplex oligonucleotide is at least 90% identical to SEQ ID NOs: 1-2, 5-10, as determined using the GCG BestFit software of the Wisconsin sequence analysis package, utilizing the Smith and Waterman algorithm, where gap weight equals 50, length weight equals 3, average match equals 10 and average mismatch equals −9.

According to still further features in the described preferred embodiments the COX-II specific small interfering duplex oligonucleotide is a single stranded oligonucleotide.

According to still further features in the described preferred embodiments the COX-II specific small interfering duplex oligonucleotide is a double stranded oligonucleotide.

According to still further features in the described preferred embodiments the pharmaceutical composition is packaged in a container and identified in print in or on the container for use in a medical condition whereby reducing COX-II expression is beneficial.

According to still further features in the described preferred embodiments the disease is selected from the group consisting of: inflammatory diseases including inflammatory bowel disease, rheumatoid arthritis, multiple sclerosis, and inflammation due to endotoxin exposure or endotoxic shock, reproductive diseases including premature labor, pre-term premature rupture of the fetal membranes (PROM), premature effacement and dilation and endometriosis, respiratory ARDS, kidney diseases including glomerulitis and glomerulonephritis, digestive diseases including chronic liver disease, ulcerative colitis, cell proliferative disorders including cancer, and neuerodegenerative diseases including Alzheimer's and Parkinson's disease and stroke and pain.

The present invention successfully addresses the shortcomings of the presently known configurations by providing oligonucleotides and methods using same for treating cox-ii associated diseases.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-c are photomicrographs exhibiting immunofluorescent detection of COX-I protein expression in BAEC cells in the absence (FIG. 1a) or presence (FIG. 1b) of PMA stimulation. FIG. 1c is a photomicrograph exhibiting COX-I negative staining in the absence of COX antibody. Magnification in all frames corresponds to 20 X.

FIGS. 2a-c are photomicrographs exhibiting immunohistochemical analysis of unstimulated (FIG. 2a) and PMA stimulated (FIG. 2b) bovine aortic endothelial cells (BAECs) stained with anti-COX-II antibody. FIG. 2c is a photomicrograph exhibiting negative staining in the absence of anti-COX-II antibody. Magnification in all frames corresponds to 20 X.

FIG. 3 is an immunoblot showing expression of COX-II in untreated and PMA stimulated BAEC cells.

FIGS. 4a-b are photomicrographs depicting uptake and distribution of BAEC cells lipofected without (FIG. 4a) or with (FIG. 4b) COX-II-specific fluorescent RNAi. Magnification in all frames corresponds to 40 X.

FIG. 5a is an immunoblot of COX-II transfected BAEC cell extracts probed for expression of COX-II (72 KD) and actin (42 KD), following transfection with or without duplex RNAi-COX-II (Lane 1) or duplex RNAi-Lac Z (Lane 4). PMA was added to the media, as indicated (Lanes 1-3) following 36 hours of duplex RNA incubation.

FIG. 5b is a standard error of the mean (mean±sem) optical density measurement for 4 independent determinations of COX-II expression under the conditions described in FIG. 5a. PMA treatment alone was used as the 100%. Note, a 36% decrease in COX-II expression (Lane 1) following incubation with duplex RNAi-COX-II as compared to untreated cells (Lane 3).

FIG. 6 is an immunoblot showing the effect of COX-II-specific duplex RNAi and LacZ-specific duplex RNAi on COX-I and II expression in BAEC cells. Lane 1—non-transfected BAEC cells in the absence of PMA treatment; Lane 2—COX-II-specific duplex RNAi transfected BAEC cells in the presence of PMA; Lane 3—non-transfected BAEC cells in the presence of PMA treatment; Lane 4—LacZ-specific duplex RNAi transfected BAEC cells in the presence of PMA; Lane 5—Bovine Seminal vesicle expression of COX-I. Each lane includes 10 μg protein.

FIGS. 7a-c are photomicrographs of immunohistochemical analysis of PMA stimulated BAEC cells transfected with duplex RNAi-COX-II (FIG. 7a, magnification is at 20 X) or duplex RNAi-Lac Z (FIG. 7b, magnification is at 20 X) and probed with anti-COX-II polyclonal antibody. Negative control for staining was effected in the absence of COX-II antibody (FIG. 7c, magnification is at 40 X). Staining was visualized with an HRP reaction product.

FIG. 8 is a graphic representation of PGE production in BAEC cells in the absence (a) or presence of PMA (b-d) with either duplex RNAi-Lac Z (c) or duplex RNAi-COX-II (b). Each column represents the mean±sem PGE concentration in media collected from at least 12 wells.

### DESCRIPTION OF THE PREFERRED EMBODIMENTS

The present invention is of novel oligonucleotides, which can be used to treat COX-II associated diseases.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Cyclooxygenase is the key enzyme required for the conversion of arachidonic acid to prostaglandins (PGs). It is classified into two isoforms, a constitutive form COX-I, which produces PGs, which regulate homeostatic functions and an inducible form COX-II. Induction of COX-II activity elicits unregulated production of prostaglandins, which production is associated with numerous medical conditions such as, hyper-proliferative diseases, pre-mature rupturing of fetal membranes and stroke.

Most of the currently available COX-II inhibitors also inhibit COX-I activity, causing gastro- and renal-toxicity and therefore their widespread use has been limited.

Exclusive inhibition of COX-II activity was also attempted using COX-II specific antisense molecules. However, these sequence specific reagents fail to efficiently inhibit COX-II activity due to poor incorporation/delivery efficiencies, low stability of the antisense reagent, non-specificity and poor availability of the target COX-II site which is associated with high occurrence of secondary structures.

As is illustrated in the examples section, which follows, the present inventor, through laborious experimentation, has provided, for the first time, a small interfering oligonucleotide duplex, which enables to inhibit COX-II (GenBank Accession No: NM000963) protein production without affecting COX-I expression.

The small interfering oligonucleotide duplex of the present invention directs sequence specific degradation of mRNA through the previously described mechanism of RNA interference (RNAi) [Hutvagner and Zamore (2002) Curr. Opin. Genetics and Development 12:225-232].

RNA interference is a two step process. The first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processively cleaves dsRNA (introduced directly or via a transgene or a virus) in an ATP-dependent manner. Successive cleavage events degrade the RNA to 19-21 bp duplexes (siRNA), each with 2-nucleotide 3′ overhangs [Hutvagner and Zamore (2002) Curr. Opin. Genetics and Development 12:225-232 and Bernstein (2001) Nature 409:363-366].

In the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore (2002) Curr. Opin. Genetics and Development 12:225-232, Hammond et al. (2001) Nat. Rev. Gen. 2:110-119, Sharp (2001) Genes. Dev. 15:485-90]. Although the mechanism of cleavage is still to be elucidated, research indicates that each RISC contains a single siRNA and an RNase [Hutvagner and Zamore (2002) Curr. Opin. Genetics and Development 12:225-232].

Because of the remarkable potency of RNAi, an amplification step within the RNAi pathway has been suggested. Amplification could occur by copying of the input dsRNAs, which would generate more siRNAs, or by replication of the siRNAs themselved. Alternatively or additionally, amplification could be effected by multiple turnover events of the RISC [Hammond et al. (2001) Nat. Rev. Gen. 2:110-119, Sharp (2001) Genes. Dev. 15:485-90, Hutvagner and Zamore (2002) Curr. Opin. Genetics and Development 12:225-232]. For more information on RNAi see the following reviews Tuschl (2001) ChemBiochem. 2:239-245, Cullen (2002) Nat. Immunol. 3:597-599 and Brantl (2002) Biochem. Biophys. Act. 1575:15-25.

As is further illustrated in the Examples section which follows and in sharp contrast to previous RNA silencing methods (e.g., antisense molecules) directed at the inhibition of COX-II [U.S. Pat. No. 6,344,323 and Stein (2001) J. Clin. Invest. 108:641-644], the small interfering oligonucleotide duplexes of the present invention mediate strong, specific, efficient and stable suppression of COX-II gene expression and as such open new avenues for COX-II specific therapy.

Thus according to one aspect of the present invention there is provided a COX-II-specific small interfering duplex oligonucleotide.

As used herein, the phrase “duplex oligonucleotide” refers to an oligonucleotide structure or mimetics thereof, which is formed by either a single self-complementary nucleic acid strand or by at least two complementary nucleic acid strands. The “duplex oligonucleotide” of the present invention can be composed of double-stranded RNA (dsRNA), a DNA-RNA hybrid, single-stranded RNA (ssRNA), isolated RNA (i.e., partially purified RNA, essentially pure RNA), synthetic RNA and recombinantly produced RNA. duplex oligonucleotide s of the present invention may be composed of naturally-occurring bases, sugars and covalent internucleoside linkages (e.g., backbone) as well as of non-naturally-occurring portions which function similarly. Such modified or substituted oligonucleotides (i.e., analogs) are often preferred over native forms because of desirable properties such as, for example, enhanced cellular uptake, enhanced affinity for nucleic acid target and increased stability in the presence of nucleases.

Preferably, the COX II specific small interfering duplex oligonucleotide of the present invention is an oligoribonucleotide composed mainly of ribonucleic acids.

The COX-II specific small interfering duplex oligonucleotide of the present invention need not display absolute homology to COX-II in order to mediate RNA interference (RNAi) [Plasterk and Ketting (2000) Curr. Opin. Genet. Dev. 10:562-7]. Rather, it must be sufficient to allow the formation of a double stranded structure under the conditions employed, with a lower threshold of about 90% identity (±10%), as determined using the GCG BestFit software of the Wisconsin sequence analysis package utilizing the Smith and Waterman algorithm, where gap weight equals 50, length weight equals 3, average match equals 10 and average mismatch equals −9. It will be appreciated though, that for optimal COX-II RNA interference, oligonucleotides of 100% identity are preferably used according to this aspect of the present invention, as a mismatch of even a single nucleotide can, at times, reduce interference efficiency [Brummelkamp (2002) Science 296:550-53].

Since introduction of long dsRNA molecules (i.e., >30 bp) into most mammalian cells causes nonspecific suppression of gene expression, which can be attributed to the interferon-mediated antiviral response, the oligonucleotide duplexes of the present invention are preferably less than 30 bp long (siRNAs), preferably between 15-30 bp, more preferably between 18-25 and most preferably between 21-23 bp.

It will be appreciated that since dsRNA induced antiviral response is absent from a number of mammalian cells including mouse embryonic stem (ES) cells oligonucleotides of the present invention can be longer than 30 bp, when introduced into such cells [Elbashir (2001) Nature 411:494-98].

Oligonucleotides of the present invention can be blunt ended or comprise at least one overhanging end (e.g., 5′, 3′). Overhanging ends can be from about 1 to about 6 nucleotides, from about 1 to about 3 nucleotides, or from about 2 to about 4 nucleotides in length.

Examples of COX-II-specific oligonucleotides suitable for use with the preset invention are provided in Table 1, below.

TABLE 1

| Oligonucleotide | | COX-II |
|---|---|---|
| Description | Oligonucleotide sequence | coordinates |
| Natural (SEQ ID's NO: 1 | augaucuacccgccucauguu | 884 |
| and 3) | uuuacuagaugggcggaguac | |
| Natural (SEQ ID NO's: 4 | ucuuauuauaccagagcucuu | 503 |
| and 5) | ggagaauaauauggucucgag | |
| Natural (SEQ ID NO's: 6 | ccagcacuucacccaucaauu | 673 |
| and 7) | cgggucgugaaguggguagu | |
| Natural (SEQ ID NO's: 8 | agacagcauaagcugcgccu | 785 |
| and 9) | ucucugucguauucgacgcgg | |

It will be appreciated that other oligonucleotides, which are not listed herein can also be employed to mediate COX-II specific interference. These can be designed and configured according to the following guidelines (www.ambion.com/techlib/tn/91/912.html).

First, COX-II mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as preferably the BLAST software available from the NCBI server (www.ncbi.nlm.nih.gov/BLAST/) or the GCG BestFit software of the Wisconsin sequence analysis package. Putative target sites which exhibit significant homology to other coding sequences are filtered out.

Qualifying target sequences are selected for synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

Oligonucleotides designed according to the teachings of the present invention are tested for their ability to reduce COX-II mRNA and protein level without affecting COX-I expression. Methods for detecting mRNA and protein levels are well known in the art and are described in Examples 1-2 of the Examples section. Oligonucleotides which meet these requirements, are further tested for their ability to reduce PGs production in transfected cells, as described in Example 3 of the Examples section which follows.

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

In vitro transcription can also be utilized for synthesizing the oligonucleotides of the present invention. Such an in-vitro transcription kit is the Silencer™ which is available from www.ambion.com.

The oligonucleotides of the present invention may comprise heterocylic nucleosides consisting of purines and the pyrimidine bases, bonded in a 3′ to 5′ phosphodiester linkage.

Preferably used oligonucleotides are those modified in either backbone, internucleoside linkages or bases, as is broadly described hereinunder. Such modifications can oftentimes facilitate oligonucleotide uptake and resistivity to intracellular conditions.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. NOs: 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention, are disclosed in U.S. Pat. No. 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Such bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications.

Another modification of the olignucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-HOURS-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

Preferably, oligonucleotides of the present invention are transcribed from expression vectors, which can facilitate stable expression of the siRNA transcripts once introduced into a host cell. These vectors are engineered to express small hairpin RNAs (shRNAs), which are processed in-vivo into siRNA molecules capable of carrying out gene-specific silencing [Brummelkamp (2002) Science 296:550-53, Paddison et al. (2002) Genes Dev. 16:948-58, Paul et al. (2002) Nature Biotech. 20:505-08, Yu et al. (2002) Proc. Natl. Acad. Sci. USA 99:6047-52].

An example for a suitable expression vector is the pSUPER™, which includes the polymerase-III H1-RNA gene promoter with a well defined start of transcription and a termination signal consisting of five thymidines in a row (T5) [Brummelkamp (2002) Science 296:550-53]. Most importantly, the cleavage of the transcript at the termination site is at a position following the second uridine, thus yielding a transcript which resembles the ends of synthetic siRNAs, which also contain nucleotide overhangs. siRNA is cloned such that it includes a COX-II sequence, separated by a short spacer from the reverse complement of the same COX-II sequence. The resulting transcript folds back on itself to form a stem-loop structure, which mediates COX-II RNAi.

Another suitable siRNA expression vector encodes the sense and antisense siRNA under the regulation of separate polIII promoters [Miyagishi and Taira[(2002) Nature Biotech. 20:497-500]. The resultant siRNA includes 5 thymidine termination signal.

Oligonucleotide sequences of the present invention can also be placed under bi-directional promoters to produce both the sense and antisense transcripts from the same promoter construct, thus simplifying the construction of expression vectors and achieving an equal molar ratio of cellular sense and antisense sequences. Examples for bi-directional promoters are disclosed in U.S. patent application Ser. No. 20,020,108,142.

Oligonucleotides of the present invention can be labeled to analyze subcellular distribution, in vivo stability, transfection efficiency, and expression attenuation of the cognate COX-II gene. It can also be used with a labeled antibody in double label experiments to correlate siRNA uptake with down-regulation of the target COX-II gene. Labeling can be effected using any radioactive, fluorescent, biological or enzymatic labels of standard use in the art. siRNA labeling kits are available from www.ambion.com.

The current state of the art did not anticipate success in the application of duplex siRNA-mediated gene silencing in mammalian systems, despite the enormous potential for the use of duplex RNA-mediated interference, in terms of low dosage required and broad host cell applicability. Yet, the present invention provides oligonucleotides which mediate strong and efficient COX-II gene silencing. As is further described in the Example 3 of the Examples section which follows, as little as 0.83 mM of COX-II specific RNAi was sufficient to reduce PGE production by 40% in BAEC cells.

The results presented herein clearly illustrate the efficacy of the oligonucleotides of the present invention in suppressing COX II activity and as such provide a novel approach for treating variety of diseases or pathological conditions associated with COX-II activity. Examples include but are not limited to inflammatory diseases including inflammatory bowel disease, rheumatoid arthritis, multiple sclerosis, and inflammation due to endotoxin exposure or endotoxic shock, reproductive diseases including premature labor, pre-term premature rupture of the fetal membranes (PROM), premature effacement and dilation and endometriosis, acute respiratory distress syndrome (ARDS), kidney diseases including glomerulitis and glomerulonephritis, digestive diseases including chronic liver disease, ulcerative colitis, cell proliferative disorders including familial adenomatous polyposis, colorectal cancer, prostate cancer, pancreatic cancer, skin cancer, head and neck cancer, esophagus cancer, breast cancer, lung cancer, and neuerodegenerative diseases including Alzheimer's and Parkinson's disease and stroke and pain. For further details on potential medical conditions which involve COX-II upregulated activity see Turini and Dubois (2002) Annu. Rev. Med. 53:35-57.

Thus, according to another aspect of the present invention there is provided a method of reducing COX-II expression in a subject in need thereof, thereby reducing COX-II-mediated prostaglandin production [e.g., (PG)Gs, PGHs, PGIs, PGAs, PGBs, PGDs, PGEs and PGFs].

Preferred subjects according to the present invention are mammals such as canines, felines, ovines, porcines, equines, bovines, humans and the like.

The method includes providing to the subject a therapeutically effective amount of the COX-II specific oligonucleotide of the present invention.

Preferably, the oligonucleotides of the present invention are administered at concentration of between, 0.1-25 μg/Kg body weight, preferably 1-25 μg/Kg body weight, more preferably 1-20 μg/Kg body weight and even more preferably 5-15 μg/Kg body weight.

The oligonucleotide (i.e., active ingredient) of the present invention can be provided to the subject per se, or as part of a pharmaceutical composition where it is mixed with a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the preparation accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media (Mutter et al. (1979).

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections.

Alternately, one may administer a preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including the preparation of the present invention formulated in a compatible pharmaceutical carrier may also be prepared, placed in an appropriate container, and labeled for treatment of an indicated condition.

Pharmaceutical compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

As is described hereinabove, the oligonucleotides of the present invention can also be expressed from a nucleic acid construct which can be administered to the subject employing any suitable mode of administration, described hereinabove (e.g., in-vivo gene therapy). Such a nucleic acid construct is introduced into a target cell or cells via appropriate gene delivery vehicle/methods (transfection, transduction, homologous recombination, etc.) and an expression system as needed and then the modified cells are expanded in culture and returned to the subject (i.e., ex-vivo gene therapy). Preferably, oligonucleotides of the present invention are introduced using transfection reagents dedicated to siRNA transfer to mammalian cells. Examples for such include but are not limited to siPORT™ Amine (i.e., a polyamine mixture) and siPORT™ Lipid (i.e., a mixture of cationic and neutral lipids).

In case the oligonucleotide of the present invention is introduced into a cell in which RNAi does not normally occur, the factors needed to mediate RNAi are introduced into such a cell or the expression of the needed factors is induced, as disclosed in U.S. patent application Ser. No. 20,020,086,356.

The oligonucleotides of the present invention can be included in a therapeutic kit. For example, siRNA oligonucleotide sets can be packaged in one or more containers with appropriate buffers and preservatives and used for directing therapeutic treatment.

Thus, the oligonucleotides can be each mixed in a single container or placed in individual containers. Preferably, the containers include a label. Suitable containers include, for example, bottles, vials, syringes, and test tubes. The containers may be formed from a variety of materials such as glass or plastic. The kit can include several distinct types of duplex oligonucleotides capable of suppressing COX II mRNA expression (corresponding to different regions within the COX II mRNA), of which all or some can be administered according to need and observed individual effect.

In addition, other additives such as stabilizers, buffers, blockers and the like may also be added.

The kit can also include instructions for determining if the tested subject is suffering from, or is at risk of developing, a condition, disorder, or disease associated with COX-II upregulation.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. HOURS. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Characterization of Cyclooxygenase Isoforms Present in BAEC Cells Material and Experimental Methods

BAEC primary cell culture preparation—Preparation of bovine aortic endothelial cell (BAEC) cultures was adapted from methods previously described [Dannhardt (2001) Inflamm. Res. 50:262-2691. Segments of bovine thoracic aortic, 30 cm in length, were taken from cows at a slaughterhouse. All subsequent operations were carried out in a laminar flow hood. Adipose tissue and fat were removed from the aorta, which was then dissected longitudinally, and fixed on a plate, intimal face upwards. The endothelial surface was incubated for 40 minutes at 37° C. in an atmosphere of 95% air and 5% CO₂ with 5 ml dispase (480 mg/100 ml H₂O, Sigma, Israel). Detached endothelial cells were then collected in a sterile 15 ml tube containing 10 ml growth culture medium (medium 199 Biological Industries, Bet Haemek, Israel) containing 20% heat-inactivated fetal calf serum (FCS), benzylpenicillin 100 U/ml, streptomycin 100 U/ml and nystatin 200 U/ml). Following centrifugation at 165×g for 10 minutes, the cell pellet was resuspended in culture medium. Bovine aortic endothelial cells (BAECs) were placed in a 25 cm² cell culture flasks and maintained in a 95% air, 5% CO₂ humidified incubator at 37° C. The culture medium was replaced every three days and changed to 10% serum culture medium. Upon reaching confluence (7-10 days), the cells were sub-cultured by detaching them with 3 ml 0.25% trypsin-EDTA solution.

Generation of duplex RNAi—Annealed fluorescent COX-II-specific dsRNA (SEQ ID NO: 1 and 2) and LacZ-specific (V00296) dsRNA (SEQ ID NO: 3 and 4) were purchased from Orbigen (www.orbigen.com).

Transfection procedure—One day prior to transfection, BAEC cells were trypsinized, diluted in fresh culture medium without antibiotics and plated in a 24 well plate such that subconflunece was achieved on the day of transfection. Transient transfection of dsRNA was effected using LipofectAMINE™ (BRL-Life Technologies) as described [Elbashir et al (2001) Nature 411:494-98]. Briefly, 3 μl of OLIGOFECTAMINE Reagent were mixed with 12 μl of Opti-MEM and incubated for 7 to 10 minutes at room temperature. Concurrently, 0.5 μg of fluorescent siRNA oligonucleotide was diluted in 50 μl of Opti-MEM. siRNA solution was then combined with the lipofectamine solution and gently agitated. Following incubation for 25 minutes at room temperature, transfection solution turned turbid. Following turbidity detection, an additional 32 μl of fresh Opti-MEM was added to obtain a final volume of 100 μl. The entire siRNA-OLIGOFECTAMINE mixture was added to a well containing the cultured cells in 500 μl medium 199. 5 hr post treatment (PT), a FCS was added to a final concentration of 5%. As indicated, PMA (phorbol 12-myristate acetate, Sigma, 20 nm) was added to the culture medium 36 hours PT. 48 hours PT transfected cells were harvested for immuno-histochemistry and western blot. Specific silencing was confirmed in at least three independent experiments.

COX-II induction—Cultured BAEC cells grown for 36 hours were incubated with 20 nM phorbol myristic acid (PMA). [Sigma, St. Louis, Mo.] Cells were incubated for additional 12 hours in the presence of PMA.

Immunohistochemistry and immunofluorescence microscopy for detection of COX expression—BAEC cells were plated on coverslips and grown in 24-well culture plates. 48 hours post treatment cells were harvested. Cells were washed with PBS and fixed with 100% methanol for 10 minutes at −20° C. Following blocking with 2% BSA blocking buffer and rehydration with PBS, cells were incubated with anti-COX-II monoclonal antibody (1:200, Santa Cruz Biotechnology, Santa Cruz) or with polyclonal anti-COX antibody [Izhar (1992) Prostaglandins 43:239-54], recognizing both COX-I and COX-II, for 60 minutes. A fluorescein isothiocyanate-labeled anti-rabbit IgG (1:50, Sigma, Israel) secondary antibody was used to detect the polyclonal anti-COX antibody while peroxidase conjugate anti-goat IgG (1:200, Sigma, Israel) was used to detect the COX-II specific antibody. A color reaction was developed using AEC (Histostain-AEC, Zymed Laboratories, South San Francisco, Calif.), and observed under a compound light microscope. Control slides were incubated with bovine serum albumin (Biological Industries, Bet Haemek, Israel) (Bein place of primary antibody, and similarly developed for detection of background staining. Photographs were taken with equal exposure times for treated and untreated cells.

Immunoblot detection of COX expression—48 hours post treatment, BAEC cells were scraped, washed once in ice-cold PBS and harvested. Cells were solubilized in 10 ml lysis buffer [4% SDS, 1.25 mM Tris 7.5, 1 mM Benzamidine 1 mM Na₃VO₄, 1 mM PMSF (phenyl-methyl-sulphonyl-fluoride)] while gently shaken. Cell extracts were centrifuged at 13,000×g for 5 minutes and boiled for 5 minutes in the presence of sample buffer (1:1 ratio, Tris 0.2 mM, SDS 8% w/v, glycerol 40% v/v, 2-mercaptoethanol 20% v/v and bromphenol blue 0.2% w/v). Equal amounts of total protein were electrophoresed through 10% SDS-PAGE, transferred onto 0.1 mm nitrocellulose membranes and probed with goat anti-COX-II polyclonal antibody ((1:200, Santa Cruz Biotechnology, Santa Cruz or polyclonal anti-COX ((1:200 made in our laboratory, Izhar et al, 1992; II]), recognizing both COX-I and COX-II. Two secondary antibodies were used for development of a reaction product: (a) Anti-goat IgG-Horseradish peroxidase (HRP, BioRAD, Hercules, Calif. (1:10,000) or (b) Anti-goat IgG peroxidase conjugate (Sigma; Israel 1:40,000). Reaction product was visualized using ECL (Enhanced Chemiluminesence) Substrate (Amersham Life Sciences, Buckinghamshire) for detection of HRP according to manufacturer's instructions. To confirm equal loading, blots were re-probed with actin antibody (1:1000; Sigma, Israel) and the same procedure for HRP staining described above was repeated.

### Experimental Results

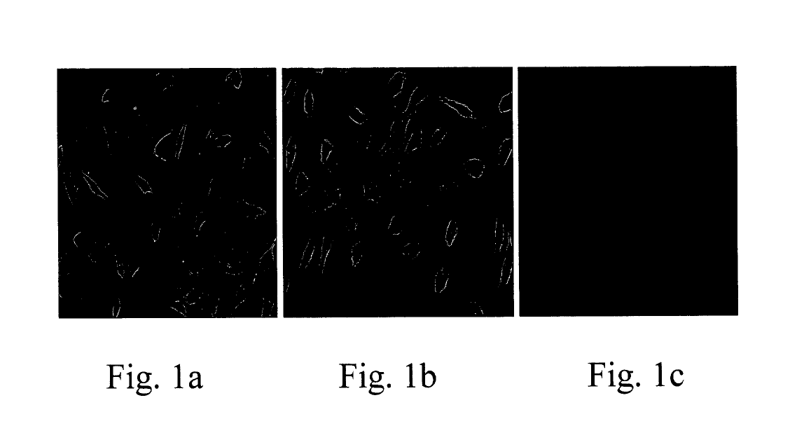

Immunohistochemical localization of cyclooxygenase isoforms present in BAEC cells—COX-I protein expression was observed in BAEC cells both in the presence or absence of PMA treatment (FIGS. 1a-b). Fluorescent immunostaining for COX-I was evident in the cytoplasm of the cells. The peri-nuclear envelope region of the cells revealed the most intense staining. Because the antibody used for immunofluorescent detection recognized both COX-I and II, PMA treated cells (FIG. 1b) demonstrated a potent color reaction compared with untreated cells (FIG. 1a). No staining was evident in negative controls (FIG. 1c).

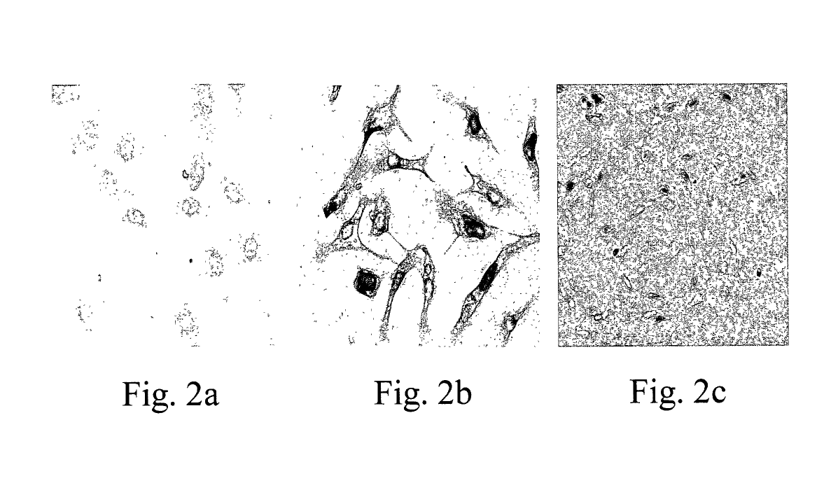

In contrast, COX-II expression was revealed only following induction with PMA stimulation (FIGS. 2a-b). While no staining of COX-II was detected in the absence of PMA pretreatment (FIG. 2a) expression was readily induced in cells following PMA stimulation (FIG. 2b). No staining was evident in negative controls (FIG. 2c).

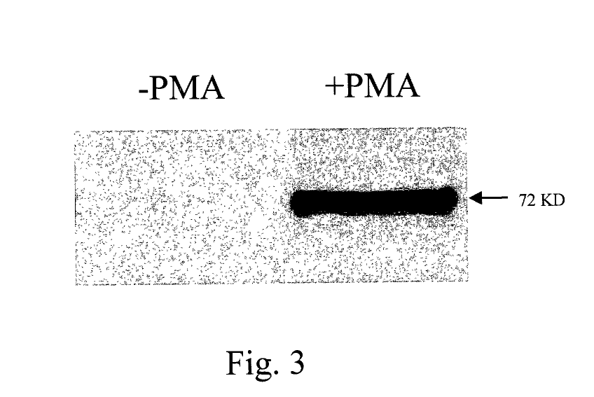

Immunoblot detection of COX-II in BAEC cells—To quantitate the level of inducible COX-II, BAEC cells were treated with PMA. As shown in FIG. 3, consistent with the immunohistochemical results (FIGS. 2a-c), western analysis of BAEC cells detected COX-II expression only following induction with PMA.

### Example 2

### Selective Inhibition of COX-II Expression in BAEC Cells Following Transfection with Duplex RNAi-COX-II

### Material and Experimental Methods

All procedures, including immunoblot, immunohistochemistry and immunofluorescence assays were conducted as described under Example 1 above.

### Experimental Results

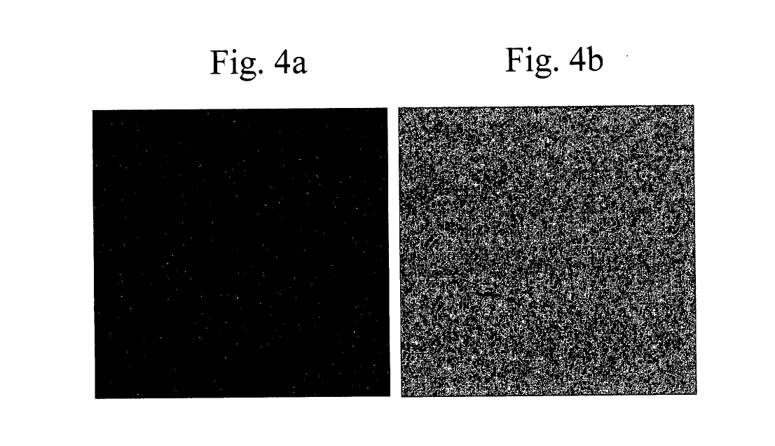

Uptake and distribution of COX-II specific RNAi in BAEC cells—To determine the efficiency of dsRNA uptake into cells, BAEC cells were lipofected without (FIG. 4a) or with (FIG. 4b) COX-II specific fluorescent RNAi. As shown in FIG. 4b, transfected cells (FIG. 4b) exhibited a green fluorescence in the majority of cells of the examined field, demonstrating the efficiency of dsRNA uptake.

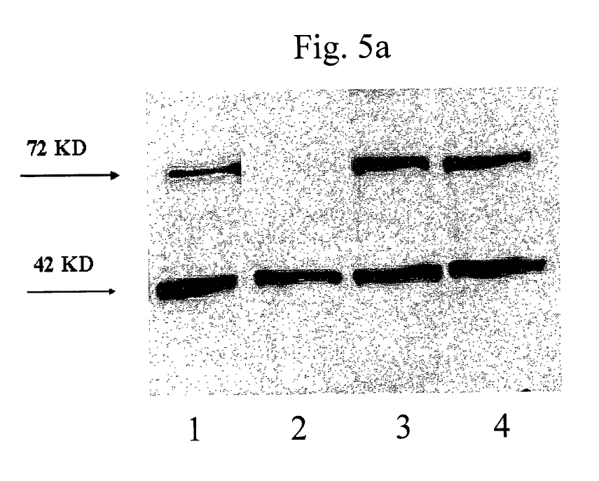

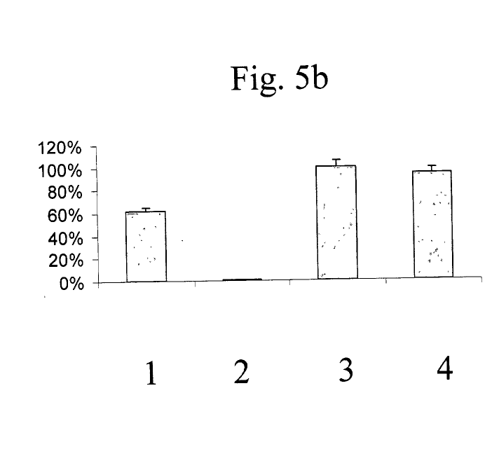

Immunoblot detection of duplex RNAi inhibition of COX-II expression in treated BAECs—COX-I and COX-II protein expression in BAEC cells following treatment with either the duplex RNAi-COX-II (SEQ ID NO: 1 and 2) or duplex RNAi-Lac Z control genes (encoding beta-galactosidase) (SEQ ID NO: 3 and 4) was determined by immunoblot analysis, probing with specific antibodies against COX-II. Western blots were performed for both treated and non-treated cells. As can be seen in FIGS. 5a-b, PMA-induced COX-II expression was inhibited (about 36%) by duplex RNAi-COX-II (0.83 mM, Lane 1), as compared to cells not treated with duplex RNAi (Lane 3).

In order to demonstrate the specificity of the duplex RNAi-COX, RNAi against Lac Z was prepared and tested in the same system. Transfection with RNAi-Lac Z (0.83 mM) (SEQ ID NO: 3 and 4) had no effect on COX-II expression in PMA treated BAECs (FIG. 5a, Lane 4). Thus duplex RNAi-COX incubation specifically inhibited PMA induced COX-II expression in BAEC cells.

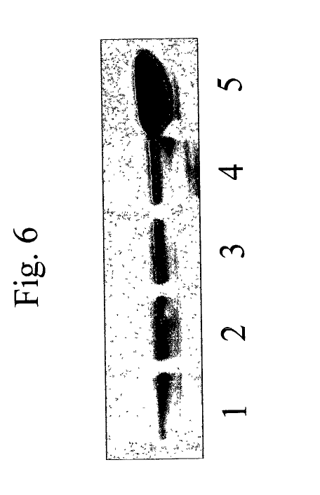

Analysis of COX-I expression by immunoblot indicated no inhibition of COX-I expression in either the presence of duplex RNAi-COX-II or dsRNA-Lac Z treated, PMA treated BAEC cells (FIG. 6).

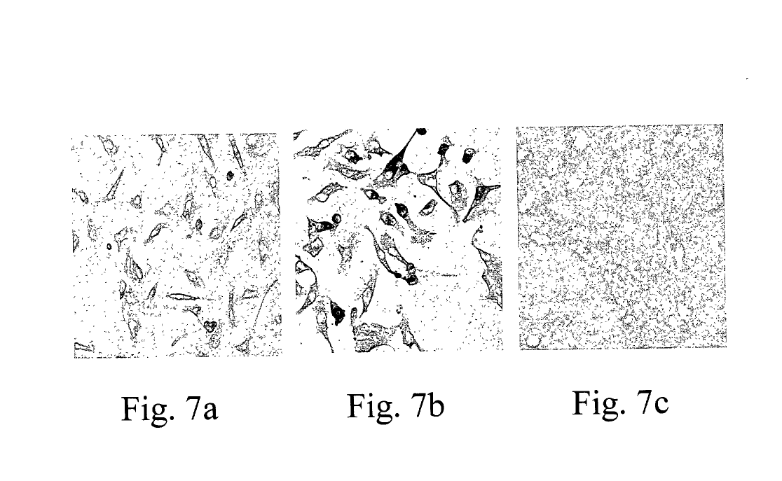

Diminished immunohistochemical localization of cyclooxygenase isoforms in duplex RNAi inhibited BAEC cells—Similarly to the immunoblot results, immunohistochemical analysis revealed COX-II inhibition following BAEC cell incubation with duplex RNAi-COX (SEQ ID NO: 1 and 2). While prominent COX-II protein expression is observed in duplex RNAi-Lac Z (SEQ ID NO: 3 and 4) treated cells (FIG. 7b) as detected by immunostaining with a COX-II specific antibody, cells treated with duplex RNAi-COX (SEQ ID NO: 1 and 2) revealed a precipitous decline in immunostaining (FIG. 7a). Negative controls revealed no background staining at all, as expected (FIG. 7c).

Thus, a duplex RNAi specific for COX-II selectively inhibited COX-II expression in BAEC cells, indicating a role for duplex RNAi-mediated inhibition in vivo.

### Example 3

### Selective Inhibition of COX-II Activity in BAEC Cells Following Transfection with COX-II-Specific siRNA

### Material and Experimental Methods

Measurement of PGE and PGF—Radioimmunoassay (RIA) detection of prostaglandin E2 (PGE2) and prostaglandin F2-α (PGF2-α) was conducted as previously described [Shemesh M et al. (1997). Endocrinology 138: 4844-85]. Briefly, 120 μl aliquots of culture media from BAECs treated as above were collected 48 hours post treatment, prior to chromatographic separation. Of note is that that antiserum against PGF2-α (Sigma Israel, Rehovot, Israel) reacts preferentially with PGF2-α but cross-reacts as well with prostaglandin F1-α (PGF1-α) (60%) and to a negligible extent (<0.1%) with prostaglandins of the A, B and E series (PGA, PGB and PGE, respectively). Furthermore, antiserum against PGE2 reacts preferentially with PGE2 but cross-reacts as well with PGE1 (20%), PGA, PGA2, PGF1-α and PGF2-α (<10%) and to a negligible extent with PGB1 and PGB2 (<0.1%). The intra-assay coefficient of variation was 11%, and the inter-assay coefficient of variance was 13% for PGE2. PGF was not detectable (<0.1 ng/2.5 million cells) in the media following any of the treatments.

### Experimental Results

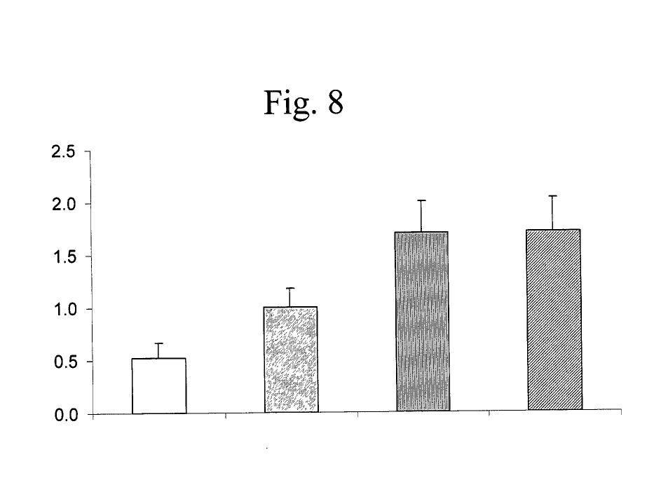

PGE production is inhibited by duplex RNAi-COX-II—PGE production was determined in BAEC culture media following a 48 hour incubation post treatment (FIG. 8). Measurement of the same sample in aliquots of 20, 50 or 100 μl indicated linearity. Media extraction with ethyl acetate (0.2 ml media/3 ml ethyl acetate) did not provide any change in PGE content as compared to that obtained with direct measurement of the media. PMA stimulation increased basal PGE production in BAECs by three-fold, regardless of the presence or absence of duplex RNAi-Lac-Z (SEQ ID NO: 3 and 4). Duplex RNAi-COX-II (SEQ ID NO: 1 and 2) inhibition of PMA stimulated BAEC COX-II expression resulted in a 40% reduction in PGE production. PGF levels were undetectable in all samples evaluated (<0.1 ng/2.5 million cells).

Notably, The marked decrease in PGE₂ production (i.e., 40%) was in good correlation with the decrease in COX-II expression following treatment with COX-II specific dsRNA (FIG. 5a).

Thus, in conclusion the dsRNA oligonucleotides of the present invention can specifically inhibit COX II expression as demonstrated by three experimental approaches: immunohistochemical staining, western blot analysis and metabolite production (i.e., PGE production). The high specificity of COX-II specific dsRNA was repeatedly demonstrated, as COX I expression was not affected by it. Furthermore dsRNA against Lac Z had no effect on the expression of either COX I or COX II.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A small interfering duplex oligonucleotide comprising a 15 to 30 base pair sequence being at least 90% identical to a contiguous nucleic acid sequence of COX-II, as determined using the GCG BestFit software of the Wisconsin sequence analysis package, utilizing the Smith and Waterman algorithm, where gap weight equals 50, length weight equals 3, average match equals 10 and average mismatch equals −9.

2. The small interfering duplex oligonucleotide of claim 1, wherein said 15 to 30 base pair sequence is selected incapable of reducing COX-I expression.

3. The small interfering duplex oligonucleotide of claim 1, wherein the small interfering duplex oligonucleotide is specifically selected capable of reducing production of at least one prostaglandin.

4. The small interfering duplex oligonucleotide of claim 3, wherein said at least one prostaglandin is selected from the group consisting of (PG)Gs, PGHs, PGIs, PGAs, PGBs, PGDs, PGEs and PGFs.

5. A pharmaceutical composition comprising the small interfering duplex oligonucleotide of claim 1 and a pharmaceutically acceptable carrier and/or diluent.

6. The small interfering duplex oligonucleotide of claim 1, wherein said 15 to 30 base pair sequence comprises at least one terminal 3′ hydroxyl group.

7. The small interfering duplex oligonucleotide of claim 1, wherein said 15 to 30 base pair sequence comprises blunt and/or overhanging ends.

8. The small interfering duplex oligonucleotide of claim 7, wherein said overhanging ends comprise ends that are 1 to 6 nucleotides in length.

9. The small interfering duplex oligonucleotide of claim 1, wherein said 15 to 30 base pair sequence comprises ribonucleotides and/or ribonucleotide analogs.

10. The small interfering duplex oligonucleotide of claim 1, wherein said 15 to 30 base pair sequence is selected from the group consisting of SEQ ID NOs: 1-2, 5-10.

11. The small interfering duplex oligonucleotide of claim 1, wherein said 15 to 30 base pair sequence comprises ribonucleotides and/or ribonucleotide analogs.

12. The small interfering duplex oligonucleotide of claim 1, wherein said 15 to 30 base pair sequence is single stranded.

13. The small interfering duplex oligonucleotide of claim 1, wherein said 15 to 30 base pair sequence is double stranded.

14. A method of reducing COX-II expression in a subject in need thereof, the method comprising administering to the subject a therapeutically effective amount of COX-II specific small interfering duplex oligonucleotide, thereby reducing COX-II expression.

15. The method of claim 14, wherein said COX-II specific small interfering duplex oligonucleotide is selected incapable of reducing COX-I expression.

16. The method of claim 14, wherein administering said COX-II specific small interfering duplex oligonucleotide concurrently or subsequently reduces a production of at least one prostaglandin.

17. The method of claim 16, wherein said at least one prostaglandin is selected from the group consisting of (PG)Gs, PGHs, PGIs, PGAs, PGBs, PGDs, PGEs and PGFs.

18. The method of claim 14, wherein said administering is effected by an administration route selected from the group consisting of intamuscular injection, intravenous injection, infusion, oral administration, topical administration, intrathecal administration, catheter-based intra-arterial administration, intravenous infusion pump administration, administration by inhalation, parenteral administration, nasal administration, rectal administration, ear administration, vaginal administration, opthalmic administration, administration via a patch device and administration via an implantable delivery device.

19. The method of claim 14, wherein said administering is effected at a concentration of said small interfering duplex oligonucleotide between 5-15 μg/Kg body weight.

20. The method of claim 14, wherein said COX-II specific small interfering duplex oligonucleotide is administered in a pharmaceutical carrier.

21. The method of claim 20, wherein said pharmaceutical carrier comprises lipomolecules.

22. The method of claim 21, wherein said lipomolecules are arranged in liposomes or micelles.

23. The method of claim 14, wherein said COX-II specific small interfering duplex oligonucleotide comprises at least one terminal 3′ hydroxyl group.

24. The method of claim 14, wherein said COX-II specific small interfering duplex oligonucleotide comprises blunt and/or overhanging ends.

25. The method of claim 24, wherein said overhanging ends comprise ends that are 1 to 6 nucleotides in length.

26. The method of claim 14, wherein said COX-II specific small interfering duplex oligonucleotide comprises ribonucleotides and/or ribonucleotide analogs.

27. The method of claim 26, wherein said COX-II specific small interfering duplex oligonucleotide is of between 15 to 30 base pairs.

28. The method of claim 27, wherein said COX-II specific small interfering duplex oligonucleotide is of between 18 to 25 base pairs.

29. The method of claim 27, wherein said COX-II specific small interfering duplex oligonucleotide is of between 21 to 23 base pairs.

30. The method of claim 14, wherein said COX-II specific small interfering duplex oligonucleotide is as set forth in SEQ ID NOs: 1-2, 5-10.

31. The method of claim 14, wherein said COX-II specific small interfering duplex oligonucleotide is at least 90% identical to SEQ ID NOs: 1-2, 5-10, as determined using the GCG BestFit software of the Wisconsin sequence analysis package, utilizing the Smith and Waterman algorithm, where gap weight equals 50, length weight equals 3, average match equals 10 and average mismatch equals −9.

32. The method of claim 14, wherein said subject has a condition selected from the group consisting of: inflammatory diseases including inflammatory bowel disease, rheumatoid arthritis, multiple sclerosis, and inflammation due to endotoxin exposure or endotoxic shock, reproductive diseases including premature labor, pre-term premature rupture of the fetal membranes (PROM), premature effacement and dilation and endometriosis, respiratory diseases including ARDS, kidney diseases including glomerulitis and glomerulonephritis, digestive diseases including chronic liver disease, ulcerative colitis, cell proliferative disorders including cancer, and neuerodegenerative diseases including Alzheimer's and Parkinson's disease and stroke and pain.

33. The method of claim 14, wherein said COX-II specific small interfering duplex oligonucleotide is a single stranded oligonucleotide.

34. The method of claim 14, wherein said COX-II specific small interfering duplex oligonucleotide is a double stranded oligonucleotide.

35. A method of reducing COX-II mediated prostaglandin production in a subject in need thereof, the method comprising administering to the subject a therapeutically effective amount of COX-II specific small interfering duplex oligonucleotide for reducing COX-II expression, thereby reducing COX-II mediated prostaglandin production.

36. The method of claim 35, wherein said COX-II specific small interfering duplex oligonucleotide is selected incapable of reducing COX-I expression.

37. The method of claim 35, wherein the prostaglandins are selected from the group consisting of (PG)Gs, PGHs, PGIs, PGAs, PGBs, PGDs, PGEs and PGFs.

38. The method of claim 35, wherein said administering is effected by an administration route selected from the group consisting of intamuscular injection, intravenous injection, infusion, oral administration, topical administration, intrathecal administration, catheter-based intra-arterial administration, intravenous infusion pump administration, administration by inhalation, parenteral administration, nasal administration, rectal administration, ear administration, vaginal administration, opthalmic administration, administration via a patch device and administration via an implantable delivery device.

39. The method of claim 35, wherein said administering is effected at a concentration of said small interfering duplex oligonucleotide between 5-15 μg/Kg body weight.

40. The method of claim 35, wherein said COX-II specific small interfering duplex oligonucleotide is administered in a pharmaceutical carrier.

41. The method of claim 40, wherein said pharmaceutical carrier comprises lipomolecules.

42. The method of claim 41, wherein said lipomolecules are arranged in liposomes or micelles.

43. The method of claim 35, wherein said COX-II specific small interfering duplex oligonucleotide comprises at least one terminal 3′ hydroxyl group.

44. The method of claim 35, wherein said COX-II specific small interfering duplex oligonucleotide comprises blunt and/or overhanging ends.

45. The method of claim 44, wherein said overhanging ends comprise ends that are 1 to 6 nucleotides in length.

46. The method of claim 35, wherein said COX-II specific small interfering duplex oligonucleotide comprises ribonucleotides and/or ribonucleotide analogs.

47. The method of claim 46, wherein said COX-II specific small interfering duplex oligonucleotide is of between 15 to 30 base pairs.

48. The method of claim 46, wherein said COX-II specific small interfering duplex oligonucleotide is of between 18 to 25 base pairs.

49. The method of claim 46, wherein said COX-II specific small interfering duplex oligonucleotide is of between 21 to 23 base pairs.

50. The method of claim 35, wherein said COX-II specific small interfering duplex oligonucleotide is as set forth in SEQ ID NOs: 1-2, 5-10.

51. The method of claim 35, wherein said COX-II specific small interfering duplex oligonucleotide is at least 90% identical to SEQ ID NOs: 1-2, 5-10, as determined using the GCG BestFit software of the Wisconsin sequence analysis package, utilizing the Smith and Waterman algorithm, where gap weight equals 50, length weight equals 3, average match equals 10 and average mismatch equals −9.

52. The method of claim 35, wherein said subject has a condition selected from the group consisting of: inflammatory diseases including inflammatory bowel disease, rheumatoid arthritis, multiple sclerosis, and inflammation due to endotoxin exposure or endotoxic shock, reproductive diseases including premature labor, pre-term premature rupture of the fetal membranes (PROM), premature effacement and dilation and endometriosis, respiratory diseases including ARDS, kidney diseases including glomerulitis and glomerulonephritis, digestive diseases including chronic liver disease, ulcerative colitis, cell proliferative disorders including cancer, and neuerodegenerative diseases including Alzheimer's and Parkinson's disease and stroke and pain.

53. The method of claim 35, wherein said COX-II specific small interfering duplex oligonucleotide is a single stranded oligonucleotide.

54. The method of claim 35, wherein said COX-II specific small interfering duplex oligonucleotide is a double stranded oligonucleotide.

55. A method of reducing COX-II expression in a subject in need thereof, the method comprising administering to the subject a therapeutically effective amount of at least one expressible polynucleotide encoding a COX-II specific small interfering duplex oligonucleotide, thereby reducing COX-II expression.

56. The method of claim 55, wherein said COX-II specific small interfering duplex oligonucleotide is selected incapable of reducing COX-I expression.

57. The method of claim 55, wherein administering said COX-II specific small interfering duplex oligonucleotide concurrently or subsequently reduces a production of at least one prostaglandin.

58. The method of claim 57, wherein said at least one prostaglandin is selected from the group consisting of (PG)Gs, PGHs, PGIs, PGAs, PGBs, PGDs, PGEs and PGFs.

59. The method of claim 55, wherein said administering is effected by an administration route selected from the group consisting of intamuscular injection, intravenous injection, infusion, oral administration, topical administration, intrathecal administration, catheter-based intra-arterial administration, intravenous infusion pump administration, administration by inhalation, parenteral administration, nasal administration, rectal administration, ear administration, vaginal administration, opthalmic administration, administration via a patch device and administration via an implantable delivery device.

60. The method of claim 55, wherein said administering is effected at a concentration of said small interfering duplex oligonucleotide between 5-15 μg/Kg body weight.

61. The method of claim 55, wherein said COX-II specific small interfering duplex oligonucleotide is administered in a pharmaceutical carrier.

62. The method of claim 61, wherein said pharmaceutical carrier comprises lipomolecules.

63. The method of claim 62, wherein said lipomolecules are arranged in liposomes or micelles.

64. The method of claim 55, wherein said COX-II specific small interfering duplex oligonucleotide comprises at least one terminal 3′ hydroxyl group.

65. The method of claim 55, wherein said COX-II specific small interfering duplex oligonucleotide comprises blunt and/or overhanging ends.

66. The method of claim 65, wherein said overhanging ends comprise ends that are 1 to 6 nucleotides in length.

67. The method of claim 55, wherein said COX-II specific small interfering duplex oligonucleotide comprises ribonucleotides and/or ribonucleotide analogs.

68. The method of claim 67, wherein said COX-II specific small interfering duplex oligonucleotide is of between 15 to 30 base pairs.

69. The method of claim 68, wherein said COX-II specific small interfering duplex oligonucleotide is of between 18 to 25 base pairs.

70. The method of claim 68, wherein said COX-II specific small interfering duplex oligonucleotide is of between 21 to 23 base pairs.

71. The method of claim 55, wherein said COX-II specific small interfering duplex oligonucleotide is as set forth in SEQ ID NOs: 1-2, 5-10.

72. The method of claim 55, wherein said COX-II specific small interfering duplex oligonucleotide is at least 90% identical to SEQ ID NOs: 1-2, 5-10, as determined using the GCG BestFit software of the Wisconsin sequence analysis package, utilizing the Smith and Waterman algorithm, where gap weight equals 50, length weight equals 3, average match equals 10 and average mismatch equals −9.

73. The method of claim 55, wherein said subject has a condition selected from the group consisting of: inflammatory diseases including inflammatory bowel disease, rheumatoid arthritis, multiple sclerosis, and inflammation due to endotoxin exposure or endotoxic shock, reproductive diseases including premature labor, pre-term premature rupture of the fetal membranes (PROM), premature effacement and dilation and endometriosis, respiratory diseases including ARDS, kidney diseases including glomerulitis and glomerulonephritis, digestive diseases including chronic liver disease, ulcerative colitis, cell proliferative disorders including cancer, and neuerodegenerative diseases including Alzheimer's and Parkinson's disease and stroke and pain.

74. The method of claim 55, wherein said COX-II specific small interfering duplex oligonucleotide is a single stranded oligonucleotide.

75. The method of claim 55, wherein said COX-II specific small interfering duplex oligonucleotide is a double stranded oligonucleotide.

76. The method of claim 55, wherein said polynucleotide encoding said COX-II specific small interfering duplex oligonucleotide is expressed from an expression vector including a promoter.

77. The method of claim 76, wherein said promoter is a bi-directional promoter.

78. A pharmaceutical composition for reducing COX-II-mediated prostaglandin production, the pharmaceutical composition comprising, as an active ingredient, a COX-II specific small interfering duplex oligonucleotide for reducing COX-II expression and COX-II-mediated prostaglandin production, and a pharmaceutically acceptable carrier.

79. The pharmaceutical composition of claim 78, wherein said COX-II specific small interfering duplex oligonucleotide is selected incapable of reducing COX-I expression.

80. The pharmaceutical composition of claim 78, wherein said prostaglandin is selected from the group consisting of (PG)Gs, PGHs, PGIs, PGAs, PGBs, PGDs, PGEs and PGFs.

81. The pharmaceutical composition of claim 78, wherein said active ingredient is effective at a concentration of said small interfering duplex oligonucleotide between 5-15 μg/Kg body weight.

82. The pharmaceutical composition of claim 78, wherein said pharmaceutically acceptable carrier comprises lipomolecules.

83. The pharmaceutical composition of claim 82, wherein said lipomolecules are arranged in liposomes or micelles. cm 84. The pharmaceutical composition of claim 78, wherein said COX-II specific small interfering duplex oligonucleotide comprises at least one terminal 3′ hydroxyl group.

85. The pharmaceutical composition of claim 78, wherein said COX-II specific small interfering duplex oligonucleotide comprises blunt and/or overhanging ends.

86. The pharmaceutical composition of claim 85, wherein said overhanging ends comprise ends that are 1 to 6 nucleotides in length.

87. The pharmaceutical composition of claim 78, wherein said COX-II specific small interfering duplex oligonucleotide comprises ribonucleotides and/or ribonucleotide analogs.

88. The pharmaceutical composition of claim 78, wherein said COX-II specific small interfering duplex oligonucleotide is of between 15 to 30 base pairs.

89. The pharmaceutical composition of claim 78, wherein said COX-II specific small interfering duplex oligonucleotide is of between 18 to 25 base pairs.

90. The pharmaceutical composition of claim 78, wherein said COX-I specific small interfering duplex oligonucleotide is of between 21 to 23 base pairs.

91. The pharmaceutical composition of claim 78, wherein said COX-II specific small interfering duplex oligonucleotide is as set forth in SEQ ID NOs: 1-2, 5-10.

92. The pharmaceutical composition of claim 78, wherein said COX-II specific small interfering duplex oligonucleotide is at least 90% identical to SEQ ID NOs: 1-2, 5-10, as determined using the GCG BestFit software of the Wisconsin sequence analysis package, utilizing the Smith and Waterman algorithm, where gap weight equals 50, length weight equals 3, average match equals 10 and average mismatch equals −9.

93. The pharmaceutical composition of claim 78, wherein said COX-II specific small interfering duplex oligonucleotide is a single stranded oligonucleotide.

94. The pharmaceutical composition of claim 78, wherein said COX-II specific small interfering duplex oligonucleotide is a double stranded oligonucleotide.

95. The pharmaceutical composition of claim 78, packaged in a container and identified in print in or on said container for use in a medical condition whereby reducing COX-II expression is beneficial.

96. A pharmaceutical composition for reducing COX-II expression, the pharmaceutical composition comprising, as an active ingredient, a COX-II specific small interfering duplex oligonucleotide selected capable of reducing COX-II expression and a pharmaceutically acceptable carrier.

97. The pharmaceutical composition of claim 96, wherein said COX-II specific small interfering duplex oligonucleotide is selected incapable of reducing COX-I expression.

98. The pharmaceutical composition of claim 96, wherein said prostaglandin is selected from the group consisting of (PG)Gs, PGHs, PGIs, PGAs, PGBs, PGDs, PGEs and PGFs.

99. The pharmaceutical composition of claim 96, wherein said active ingredient is effective at a concentration of said small interfering duplex oligonucleotide between 5-15 μg/Kg body weight.

100. The pharmaceutical composition of claim 96, wherein said pharmaceutically acceptable carrier comprises lipomolecules.

101. The pharmaceutical composition of claim 100, wherein said lipomolecules are arranged in liposomes or micelles.

102. The pharmaceutical composition of claim 96, wherein said COX-II specific small interfering duplex oligonucleotide comprises at least one terminal 3′ hydroxyl group.

103. The pharmaceutical composition of claim 96, wherein said COX-II specific small interfering duplex oligonucleotide comprises blunt and/or overhanging ends.

104. The pharmaceutical composition of claim 103, wherein said overhanging ends comprise ends that are 1 to 6 nucleotides in length.

105. The pharmaceutical composition of claim 96, wherein said COX-II specific small interfering duplex oligonucleotide comprises ribonucleotides and/or ribonucleotide analogs.

106. The pharmaceutical composition of claim 96, wherein said COX-II specific small interfering duplex oligonucleotide is of between 15 to 30 base pairs.

107. The pharmaceutical composition of claim 96, wherein said COX-II specific small interfering duplex oligonucleotide is of between 18 to 25 base pairs.

108. The pharmaceutical composition of claim 96, wherein said COX-II specific small interfering duplex oligonucleotide is of between 21 to 23 base pairs.

109. The pharmaceutical composition of claim 96, wherein said COX-II specific small interfering duplex oligonucleotide is as set forth in SEQ ID NOs: 1-2, 5-10.

110. The pharmaceutical composition of claim 96, wherein said COX-II specific small interfering duplex oligonucleotide is at least 90% identical to SEQ ID NOs: 1-2, 5-10, as determined using the GCG BestFit software of the Wisconsin sequence analysis package, utilizing the Smith and Waterman algorithm, where gap weight equals 50, length weight equals 3, average match equals 10 and average mismatch equals −9.

111. The pharmaceutical composition of claim 96, wherein said COX-II specific small interfering duplex oligonucleotide is a single stranded oligonucleotide.

112. The pharmaceutical composition of claim 96, wherein said COX-II specific small interfering duplex oligonucleotide is a double stranded oligonucleotide.

113. The pharmaceutical composition of claim 96, packaged in a container and identified in print in or on said container for use in a medical condition whereby reducing COX-II expression is beneficial.

114. Use of COX-II specific small interfering duplex oligonucleotides for the manufacture of a medicament for the treatment and/or prevention of a medical condition whereby reducing COX-II expression is beneficial.

115. The use of COX-II specific small interfering duplex oligonucleotides of claim 114, wherein said COX-II specific small interfering duplex oligonucleotide is selected incapable of reducing COX-I expression.

116. The use of COX-II specific small interfering duplex oligonucleotides of claim 114, said small interfering duplex oligonucleotide is effective at a concentration of between 5-15 μg/Kg body weight.

117. The use of COX-II specific small interfering duplex oligonucleotides of claim 114, wherein said COX-II specific small interfering duplex oligonucleotide comprises at least one terminal 3′ hydroxyl group.

118. The use of COX-II specific small interfering duplex oligonucleotides of claim 114, wherein said COX-II specific small interfering duplex oligonucleotide comprises blunt and/or overhanging ends.

119. The use of COX-II specific small interfering duplex oligonucleotides of claim 114, wherein said overhanging ends comprise ends that are 1 to 6 nucleotides in length.

120. The use of COX-II specific small interfering duplex oligonucleotides of claim 114, wherein said COX-II specific small interfering duplex oligonucleotide comprises ribonucleotides and/or ribonucleotide analogs.

121. The use of COX-II specific small interfering duplex oligonucleotides of claim 114, wherein said COX-II specific small interfering duplex oligonucleotide is of between 15 to 30 base pairs.

122. The use of COX-II specific small interfering duplex oligonucleotides of claim 114, wherein said COX-II specific small interfering duplex oligonucleotide is of between 18 to 25 base pairs.

123. The use of COX-II specific small interfering duplex oligonucleotides of claim 114, wherein said COX-II specific small interfering duplex oligonucleotide is of between 21 to 23 base pairs.

124. The use of COX-II specific small interfering duplex oligonucleotides of claim 114, wherein said COX-II specific small interfering duplex oligonucleotide is as set forth in SEQ ID NOs: 1-2, 5-10.

125. The use of COX-II specific small interfering duplex oligonucleotides of claim 114, wherein said COX-II specific small interfering duplex oligonucleotide is at least 90% identical to SEQ ID NOs: 1-2, 5-10, as determined using the GCG BestFit software of the Wisconsin sequence analysis package, utilizing the Smith and Waterman algorithm, where gap weight equals 50, length weight equals 3, average match equals 10 and average mismatch equals −9.

126. The use of COX-II specific small interfering duplex oligonucleotides of claim 114, wherein said COX-II specific small interfering duplex oligonucleotide is a single stranded oligonucleotide.

127. The use of COX-II specific small interfering duplex oligonucleotides of claim 114, wherein said COX-II specific small interfering duplex oligonucleotide is a double stranded oligonucleotide.

128. The use of COX-II specific small interfering duplex oligonucleotides of claim 114, wherein said COX-II specific small interfering duplex oligonucleotide reducing COX-II expression concurrently or subsequently reduces prostaglandin production.

129. The use of COX-II specific small interfering duplex oligonucleotides of claim 128, wherein said prostaglandins comprise prostaglandin (PG)Gs, PGHs, PGIs, PGAs, PGBs, PGDs, PGEs and PGFs.

130. The use of COX-II specific small interfering duplex oligonucleotides of claim 114, wherein said medical condition is selected from the group consisting of: inflammatory diseases including inflammatory bowel disease, rheumatoid arthritis, multiple sclerosis, and inflammation due to endotoxin exposure or endotoxic shock, reproductive diseases including premature labor, pre-term premature rupture of the fetal membranes (PROM), premature effacement and dilation and endometriosis, respiratory ARDS, kidney diseases including glomerulitis and glomerulonephritis, digestive diseases including chronic liver disease, ulcerative colitis, cell proliferative disorders including cancer, and neuerodegenerative diseases including Alzheimer's and Parkinson's disease and stroke and pain.

131. Use of COX-II specific small interfering duplex oligonucleotides for the evaluation of a COX-II based regimen in an animal model of disease, whereby reducing COX-II expression mitigates or abolishes the onset or severity of said disease.

132. The use of COX-II specific small interfering duplex oligonucleotides of claim 131, wherein said COX-II specific small interfering duplex oligonucleotide is selected incapable of reducing COX-I expression.

133. The use of COX-II specific small interfering duplex oligonucleotides of claim 131, said small interfering duplex oligonucleotide is effective at a concentration of between 5-15 μg/Kg body weight.

134. The use of COX-II specific small interfering duplex oligonucleotides of claim 131, wherein said COX-II specific small interfering duplex oligonucleotide comprises at least one terminal 3′ hydroxyl group.

135. The use of COX-II specific small interfering duplex oligonucleotides of claim 131, wherein said COX-II specific small interfering duplex oligonucleotide comprises blunt and/or overhanging ends.

136. The use of COX-II specific small interfering duplex oligonucleotides of claim 131, wherein said overhanging ends comprise ends that are 1 to 6 nucleotides in length.

137. The use of COX-II specific small interfering duplex oligonucleotides of claim 131, wherein said COX-II specific small interfering duplex oligonucleotide comprises ribonucleotides and/or ribonucleotide analogs.

138. The use of COX-II specific small interfering duplex oligonucleotides of claim 131, wherein said COX-II specific small interfering duplex oligonucleotide is of between 15 to 30 base pairs.

139. The use of COX-II specific small interfering duplex oligonucleotides of claim 131, wherein said COX-II specific small interfering duplex oligonucleotide is of between 18 to 25 base pairs.

140. The use of COX-II specific small interfering duplex oligonucleotides of claim 131, wherein said COX-II specific small interfering duplex oligonucleotide is of between 21 to 23 base pairs.

141. The use of COX-II specific small interfering duplex oligonucleotides of claim 131, wherein said COX-II specific small interfering duplex oligonucleotide is as set forth in SEQ ID NOs: 1-2, 5-10.

142. The use of COX-II specific small interfering duplex oligonucleotides of claim 131, wherein said COX-II specific small interfering duplex oligonucleotide is at least 90% identical to SEQ ID NOs: 1-2, 5-10, as determined using the GCG BestFit software of the Wisconsin sequence analysis package, utilizing the Smith and Waterman algorithm, where gap weight equals 50, length weight equals 3, average match equals 10 and average mismatch equals −9.

143. The use of COX-II specific small interfering duplex oligonucleotides of claim 131, wherein said COX-II specific small interfering duplex oligonucleotide is a single stranded oligonucleotide.

144. The use of COX-II specific small interfering duplex oligonucleotides of claim 131, wherein said COX-II specific small interfering duplex oligonucleotide is a double stranded oligonucleotide.

145. The use of COX-II specific small interfering duplex oligonucleotides of claim 131, wherein said COX-II specific small interfering duplex oligonucleotide reducing COX-II expression concurrently or subsequently reduces prostaglandin production.

146. The use of COX-II specific small interfering duplex oligonucleotides of claim 146, wherein said prostaglandins comprise prostaglandin (PG)Gs, PGHs, PGIs, PGAs, PGBs, PGDs, PGEs and PGFs.

147. The use of COX-II specific small interfering duplex oligonucleotides of claim 131, wherein said disease is selected from the group consisting of: inflammatory diseases including inflammatory bowel disease, rheumatoid arthritis, multiple sclerosis, and inflammation due to endotoxin exposure or endotoxic shock, reproductive diseases including premature labor, pre-term premature rupture of the fetal membranes (PROM), premature effacement and dilation and endometriosis, respiratory ARDS, kidney diseases including glomerulitis and glomerulonephritis, digestive diseases including chronic liver disease, ulcerative colitis, cell proliferative disorders including cancer, and neuerodegenerative diseases including Alzheimer's and Parkinson's disease and stroke and pain.
